(19) United States
(12) Patent Application Publication
(10) Pub. No.: US 2019/0262244 A1
(43) Pub. Date: Aug. 29, 2019

(54) Whitening Oral Care Compositions

(71) Applicant: Colgate-Palmolive Company, New York, NY (US)

(72) Inventors: Dennis ONTUMI, Easton, PA (US); Venda P. MALONEY, Piscataway, NJ (US); Suman K. CHOPRA, Monroe, NJ (US); Prakasarao MANDADI, Flemington, NJ (US)

(73) Assignee: Colgate-Palmolive Company, New York, NY (US)

(21) Appl. No.: 16/411,498

(22) Filed: May 14, 2019

Related U.S. Application Data
(63) Continuation of application No. 15/107,597, filed on Jun. 23, 2016.

(51) Int. Cl. A61K 8/22; A61K 8/24; A61K 8/81; A61K 8/86; A61Q 11/00; A61K 8/90

(52) U.S. Cl. CPC A61K 8/22; A61K 8/24; A61K 8/8176; A61K 2800/596; A61Q 11/00; A61K 8/90; A61K 2800/594; A61K 8/86

(57) ABSTRACT

Described herein are oral care compositions comprising a crosslinked polyvinylpyrrolidone complexed with hydrogen peroxide; including some embodiments which further comprise an ethylene oxide, propylene oxide block co-polymer.

### BACKGROUND

Many individuals are dissatisfied with their current tooth color. Thus, there is a desire for whiter teeth and one means to achieve whiter teeth is the use of tooth whitening products.

The teeth can become discolored by foods, drinks and tobacco use. Dental stains can be classified as either extrinsic, which occur on the outer surface of teeth, or intrinsic, which occur below the surface of enamel. Most abrasive containing toothpaste remove extrinsic stains. Hydrogen peroxide can bleach both extrinsic and intrinsic stains and so provides faster and superior whitening efficacy. The peroxide can bleach the teeth, remove stains, and kill cariogenic bacteria. However, peroxide compounds are highly reactive, and consequently difficult to formulate. Moreover, hydrogen peroxide can spontaneously decompose to form oxygen gas (O₂) and water, so that on storage, the dentifrice containers may bloat, burst or leak, and the remaining formulation will not have enough peroxide remaining to clean and whiten teeth effectively. Some dentifrices initially comprise very high levels of peroxide, which decomposes over time, so that the exact amount of peroxide delivered on application is variable and largely depends on how long and under what conditions the dentifrice has been stored.

Some formulations are known that provide a form of hydrogen peroxide stabilized with crosslinked polyvinylpyrrolidone (“PVP”); however, the examples disclosed contain 5.5% by weight hydrogen peroxide/PVP complex (see US patent application 2013/0287710).

For regulatory reasons in some countries hydrogen peroxide that can be added to dentifrices is limited, for example no higher than 0.1%.

There is thus a need for low level, stable, hydrogen peroxide oral composition which provides whitening efficacy that is greater than an abrasive toothpaste.

### BRIEF SUMMARY

In some embodiments, the present invention provides oral care compositions that are stable during long term storage and remain effective to clean and whiten teeth. In some embodiments, the invention provides an oral care composition, such as a dentifrice, comprising a whitening complex comprising a crosslinked polyvinylpyrrolidone complexed with hydrogen peroxide (“PVP-H₂O₂”), wherein the amount of hydrogen peroxide in the composition is 0.01 to 0.3% by weight of the composition.

In other embodiments, the invention provides an oral care composition comprising (i) a whitening complex comprising a crosslinked polyvinylpyrrolidone complexed with hydrogen peroxide wherein the amount of hydrogen peroxide is 0.01 to 0.3% by weight, and (ii) an ethylene oxide, propylene oxide block co-polymer of formula (ethylene oxide)ₓ-(propylene oxide)_{y} wherein x is an integer of 80-150 and y is an integer 30-80, having an average molecular weight of greater than 5000 Da.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION

The following description of the preferred embodiment(s) is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The term “wt %” is an abbreviation for weight percent. The amounts given are based on the active weight of the material.

In some embodiments the compositions of the invention further comprise an abrasive. In some embodiments, the abrasive is a calcium abrasive.

By exposure to aqueous environments, as in the oral cavity, the PVP-H₂O₂ dissociates into individual species (PVP polymer and H₂O₂). The PVP-H₂O₂ complex is generally comprised of about 80% by weight polyvinyl pyrrolidone and 20% by weight H₂O₂. Single phase whitening dentifrice compounds comprising PVP-H₂O₂ complexes are described, e.g., in WO/2007/037961, and its parent US Pub. No. US 2007-0071695 A1, the contents of which are incorporated herein by reference.

It has been surprisingly discovered that a low concentration of hydrogen peroxide, e.g., 0.01 to 0.3 wt %, in one embodiment 0.05 to 0.2 wt %, in another embodiment 0.075 to 0.15%, in another embodiment about 0.1%, in a dentifrice or mouthwash, can achieve whitening effects to teeth comparable to or better than convention whitening products containing certain abrasives.

Accordingly, the invention provides a dentifrice comprising (i) a crosslinked polyvinylpyrrolidone complexed with hydrogen peroxide wherein the composition comprises 0.01 to 0.5% hydrogen peroxide, and, in one embodiment, (ii) an ethylene oxide, propylene oxide block co-polymer of formula (ethylene oxide)ₓ-(propylene oxide)_{y} wherein x is an integer of 80-150, e.g. 100-130, e.g. about 118, and y is an integer 30-80, e.g. about 60-70, e.g. about 66, having an average molecular weight of greater than 5000, e.g., 8000-13000 Da, e.g. about 9800. In some embodiments the ethylene oxide, propylene oxide co-polymer is substantially free of an ethylene oxide, propylene oxide block co-polymer of average molecular weight less than 5000 Da. An example of a suitable commercially available ethylene oxide, propylene oxide co-polymer is PLURACARE® L1220 (available from BASF, Wyandotte, Mich., United States of America).

In some embodiments, the invention provides a dentifrice comprising an abrasive, e.g., a calcium abrasive, in particular calcium pyrophosphate. In other embodiments, the invention provides an abrasive-free gel.

For example, the invention provides Composition 1, an oral composition such as a dentifrice comprising (i) a whitening complex comprising crosslinked polyvinylpyrrolidone complexed with hydrogen peroxide, wherein the amount of hydrogen peroxide in the composition is 0.01 to 0.3 wt %, e.g.:

1.1 Composition 1 wherein said composition further comprises (iii) an ethylene oxide, propylene oxide block co-polymer of formula (ethylene oxide)ₓ-(propylene oxide)_{y} wherein x is an integer of 80-150, e.g. 100-130, e.g. about 118, and y is an integer 30-80, e.g. about 60-70, e.g. about 66, having an average molecular weight of greater than 5000, e.g., 8000-13000 Da, e.g. about 9800, in an amount, e.g., of 0.01 to 15 wt %, or 5 to 12 wt %, or about 7.5 wt %.

1.2 Composition 1 or 1.1 wherein the whitening complex contains about 10-30 wt %, e.g., 15-25 wt %, for example about 17-22 wt % of hydrogen peroxide by weight, and about 5-15 wt %, for example about 7-12 wt % total nitrogen by weight; for example, having substantially the same specifications as Polyplasdone® XL-10, e.g., Polyplasdone® XL-10F, e.g., available from International Specialty Products (Wayne, N.J.);

1.3 Any of the foregoing compositions further comprising a calcium abrasive wherein the calcium abrasive comprises a calcium phosphate salt, e.g., calcium pyrophosphate, dicalcium orthophosphate dihydrate, tricalcium phosphate, and calcium polymetaphosphate;

1.4 The immediately foregoing composition wherein the calcium abrasive comprises calcium pyrophosphate;

1.5 Composition 1.3 wherein the calcium abrasive comprises calcium carbonate; 1.6 Any of the foregoing compositions wherein the total amount of hydrogen peroxide by weight of the composition is 0.05 to 0.2%, or 0.075 to 0.15%, or about 0.1%;

1.7 Any of the foregoing compositions which contains less than 2 wt % water, e.g., less than 1 wt % water, e.g., is substantially anhydrous;

1.8 Any of the foregoing compositions comprising polymer thickeners selected from (i) polyethylene glycol, (ii) polyethylene glycol—polypropylene glycol block co-polymers having a molecular weight of at least 5000, and (iii) combinations thereof;

1.9 Any of the foregoing cChecompositions additionally comprising polyethylene glycol of average molecular weight 400 to 800, e.g., about 600 Da;

1.10 Any of the foregoing compositions additionally comprising humectants, e.g. selected from glycerin, propylene glycol or a combination thereof;

1.11 Any of the foregoing compositions additionally comprising a tartar control agent, e.g., selected from tetrasodium pyrophosphate (TSPP) and sodium acid pyrophosphate (SAPP);

1.12 Any of the foregoing compositions additionally comprising a surfactant, e.g., sodium lauryl sulfate (SLS);

1.13 Any of the foregoing compositions additionally comprising an antibacterial agent, e.g., triclosan;

1.14 Any of the foregoing compositions additionally comprising an antioxidant, e.g., butylated hydoxytoluene (BHT);

1.15 Any of the foregoing compositions comprising any or all of the following ingredient classes and/or particular ingredients by weight:

| Humectants, e.g. Glycerin | 10-60%, or 20-50%, e.g., about 35% |
|---|---|
| Propylene glycol | 0.01-60%, or 5-25% e.g., about 15% |
| Thickeners, e.g., Fumed silica | 0-3%, e.g., about 1.75% |
| Polymers, e.g., Ethylene oxide, | 0.01-15%, or 5-12%, e.g., about 7.5% |
| propylene oxide block co-polymer, avg. | |
| MW > 5 kDa | |
| Polyethylene glycol 600 | 0-15% or 1-15%, e.g., about 6% |
| Polyvinylpyrrolidone | 0-10% or 1-10%, e.g., 5.75% |
| Optionally, additional linear and/or | 0.25-10 wt % |
| crosslinked polyvinylpyrrolidone | |
| Crosslinked polyvinylpyrrolidone | 0.055-1.65%, or 0.275-1.1%, or, 0.4125- |
| complexed with 15-25% hydrogen | 0.825%, e.g., about 0.55% |
| peroxide | |
| Abrasive, e.g., Calcium pyrophosphate | 0.01-45%, or about 5-30%, e.g., about 20% |
| Fluoride, Sodium monofluorophosphate | 0-2% or about 0.1-1.5%, e.g. about 1.1% |
| Surfactant, e.g., SLS | 0-3%, or 0.1-3%, e.g., about 2% |
| Tartar control agent, e.g. TSPP | 0.01-5%, or 0.1-4% e.g., about 2% |
| Antioxidant, e.g. BHT | 0.01-5%, e.g., about 0.03% |
| Flavorings | 0.1-5, e.g., 1.4% |
| Phosphoric acid | 0.01-3%, e.g., 0.2% |
| Water | <3% |

1.16 The composition resulting from the combination of the preceding ingredients;

1.17 Any of the foregoing compositions in the form of a mouthwash or toothpaste.

In another embodiment, the invention provides a dentifrice comprising a crosslinked polyvinylpyrrolidone complexed with hydrogen peroxide, together with additional linear and/or crosslinked polyvinylpyrrolidone, and a dentifrice carrier.

In some embodiments, the present invention provides oral care compositions comprising: a crosslinked polyvinylpyrrolidone complexed with hydrogen peroxide, and a stabilizing amount of an additional linear and/or crosslinked polyvinylpyrrolidone, an abrasive such as calcium pyrophosphate, and a humectant.

Some embodiments provide oral care compositions comprising: from about 0.055 to about 1.65%, by weight, crosslinked polyvinylpyrrolidone complexed with hydrogen peroxide. Other embodiments provide oral care compositions comprising: from about 0.275 to about 1.1%, by weight, crosslinked polyvinylpyrrolidone complexed with hydrogen peroxide. Still other embodiments provide oral care compositions comprising: from about 0.4125 to about 0.825%, by weight, crosslinked polyvinylpyrrolidone complexed with hydrogen peroxide. Yet other embodiments provide oral care compositions comprising: about 0.55%, by weight, crosslinked polyvinylpyrrolidone complexed with hydrogen peroxide.

In some embodiments, the present invention provides oral care compositions comprising from about 1 to about 20% of an additional linear and/or crosslinked polyvinylpyrrolidone. Some embodiments provide compositions comprising from about 1 to about 15%, by weight, of an additional linear and/or crosslinked polyvinylpyrrolidone. Some embodiments provide compositions comprising from about 1 to about 10%, by weight, of an additional linear and/or crosslinked polyvinylpyrrolidone. Some embodiments provide compositions comprising from about 5 to about 15%, by weight, of an additional linear and/or crosslinked polyvinylpyrrolidone. Other embodiments provide compositions comprising from about 7 to about 12%, by weight, of an additional linear and/or crosslinked polyvinylpyrrolidone. Further embodiments provide oral care compositions comprising from about 8 to about 11%, by weight, of an additional linear and/or crosslinked polyvinylpyrrolidone. Still further embodiments provide compositions comprising from about 8.5 to about 10%, by weight, of an additional linear and/or crosslinked polyvinylpyrrolidone. Still other embodiments provide oral care compositions comprising 9.9% or 10%, by weight, of an additional linear and/or crosslinked polyvinylpyrrolidone. Yet other embodiments provide oral care compositions comprising about 9%, by weight, of an additional linear and/or crosslinked polyvinylpyrrolidone.

In addition to the PVP/H₂O₂ complex, the compositions of the invention comprise an orally acceptable carrier, which includes all ingredients other than the PVP/H₂O₂ complex.

Some embodiments of the present invention provide a toothpaste or gel-based peroxide compositions further comprise a calcium abrasive, in particular calcium pyrophosphate. In some embodiments, the compositions comprise from about 9 to about 25%, by weight, propylene glycol. In some embodiments, the compositions comprise from about 14 to about 32%, by weight, glycerin. In other embodiments, the compositions comprise less than 20%, by weight, of a calcium abrasive. Some embodiments provide compositions comprising from about 9 to about 25%, by weight, propylene glycol; from about 14 to about 32%, by weight, glycerin; and less than 20%, by weight, of a calcium abrasive.

Still other embodiments provide oral care compositions comprising from about 20 to about 60%, by weight, humectant.

Yet further embodiments provide oral care compositions comprising from about 5 to about 25%, by weight, abrasive.

The compositions of the invention a “low water” content, meaning that a total concentration of water, including any free water and all water contained in any ingredients, is less than about 5%, preferably less than 3%, preferably less than 2% water.

Where abrasives are present, the average particle size is generally about 0.1 to about 30 microns, for example about 1 to about 20 or about 5 to about 15 microns.

In various embodiments of the present invention, the oral composition comprises an anticalculus (tartar control) agent. Generally, tartar control agents are categorized as being incompatible with some whitening agents, but embodiments of the present invention incorporate tartar control agents and whitening agents in a single phase whitening composition. Suitable anticalculus agents include without limitation phosphates and polyphosphates (for example pyrophosphates), polyaminopropanesulfonic acid (AMPS), hexametaphosphate salts, zinc citrate trihydrate, polypeptides, polyolefin sulfonates, polyolefin phosphates, diphosphonates. In some embodiments the anticalculus agent is present at about 0.1% to about 30%. The oral composition may include a mixture of different anticalculus agents. In one preferred embodiment, tetrasodium pyrophosphate (TSPP) and/or sodium acid pyrophosphate (SAPP) are used. In the one embodiment, the anticalculus agent comprises TSPP at about 1-2% and SAPP at about 0.5 to 5%. In a second preferred embodiment, tetrasodium pyrophosphate (TSPP) and/or sodium tripolyphosphate (STPP) are used. In the second preferred embodiment, the anticalculus agent comprises TSPP at about 1-2% and STPP at about 7% to 10%.

The oral care composition can optionally include at least one orally acceptable source of fluoride ions. Any known or to be developed in the art may be used. Suitable sources of fluoride ions include fluoride, monofluorophosphate and fluorosilicate salts. One or more fluoride ion-releasing compound is optionally present in an amount providing a total of about 100 to about 20,000 ppm, about 200 to about 5,000 ppm, or about 500 to about 2,500 ppm, fluoride ions.

The compositions of the invention may also comprise various dentifrice ingredients to adjust the rheology and feel of the composition such as humectants, surface active agents, thickening or gelling agents, etc.

The compositions of the present invention may comprise a surface active agent (surfactant). Suitable surfactants include without limitation water-soluble salts of C₈₋₂₀ alkyl sulfates, sulfonated monoglycerides of C₈₋₂₀ fatty acids, sarcosinates, taurates, sodium lauryl sulfate, sodium cocoyl monoglyceride sulfonate, sodium lauryl sarcosinate, sodium lauryl isoethionate, sodium laureth carboxylate and sodium dodecyl benzenesulfonate, and cocoamidopropyl betaine.

The compositions of the present invention optionally comprise a thickener. Any orally acceptable thickening agent can be used, including without limitation carbomers, also known as carboxyvinyl polymers, carrageenans, also known as Irish moss and more particularly—carrageenan (iota-carrageenan), high molecular weight polyethylene glycols (such as CARBOWAX®, available from The Dow Chemical Company), cellulosic polymers such as hydroxyethylcellulose, carboxymethylcellulose (CMC) and salts thereof, e.g., CMC sodium, natural gums such as karaya, xanthan, gum arabic and tragacanth, colloidal magnesium aluminum silicate, and colloidal and/or fumed silica and mixtures of the same. One or more thickening agents are optionally present in a total amount of about 0.1% to about 90%, for example about 1% to about 50% or about 5% to about 35%.

In various preferred embodiments, the compositions may comprise polymers and/or copolymers of polyethylene glycol, of ethylene oxide/propylene oxide, and of silicone. If such copolymers/polymers are used, they may be selected from commercially available materials. In one embodiment such block copolymer is an ethylene oxide, propylene oxide block co-polymer of formula (ethylene oxide)ₓ-(propylene oxide)_{y} wherein x is an integer of 80-150, e.g. 100-130, e.g. about 118, and y is an integer 30-80, e.g. about 60-70, e.g. about 66, having an average molecular weight of greater than 5000, e.g., 8000-13000 Da, e.g. about 9800. Block copolymers of ethylene oxide/propylene oxide are useful, but higher molecular weight, e.g., >5000 Da are preferred, e.g. including PLURACARE® L1220 (available from BASF, Wyandotte, Mich., United States of America). Low or medium molecular weight polyethylene glycol, e.g., PEG 400, PEG 600, PEG 800, PEG 1000 and mixtures thereof are also useful.

It is preferred that the carrier(s) provide a dentifrice with a viscosity of about 10,000 CPS to about 700,000 CPS, preferably about 30,000 CPS to about 300,000 CPS.

As recognized by one of skill in the art, the oral compositions of the present invention optionally include other materials, such as for example, anti-caries agents, desensitizing agents, viscosity modifiers, diluents, surface active agents, such as surfactants, emulsifiers, and foam modulators, pH modifying agents, abrasives, in addition to those listed above, humectants, mouth feel agents, sweetening agents, flavor agents, colorants, preservatives, and combinations thereof. It is understood that while general attributes of each of the above categories of materials may differ, there may be some common attributes and any given material may serve multiple purposes within two or more of such categories of materials. Preferably, the carrier is selected for compatibility with other ingredients of the composition.

Flavorants, sweeteners, colorants, foam modulators, mouth-feel agents and others additively may be included if desired, in the composition.

The compositions of the present invention optionally comprise one or more further active material(s), which is operable for the prevention or treatment of a condition or disorder of hard or soft tissue of the oral cavity, the prevention or treatment of a physiological disorder or condition, or to provide a cosmetic benefit.

The compositions may include a stannous ion or a stannous ion source. Suitable stannous ion sources include without limitation stannous fluoride, other stannous halides such as stannous chloride dihydrate, stannous pyrophosphate, organic stannous carboxylate salts such as stannous formate, acetate, gluconate, lactate, tartrate, oxalate, malonate and citrate, stannous ethylene glyoxide and the like. One or more stannous ion sources are optionally and illustratively present in a total amount of about 0.01% to about 10%, for example about 0.1% to about 7% or about 1% to about 5%.

The compositions of the present invention optionally comprise an antimicrobial (e.g., antibacterial) agent. A further illustrative list of useful antibacterial agents is provided in such as those listed in U.S. Pat. No. 5,776,435 to Gaffar et al., the contents of which are incorporated herein by reference. One or more antimicrobial agents are optionally present in an antimicrobial effective total amount, typically about 0.05% to about 10%, for example about 0.1% to about 3%.

The compositions of the present invention optionally comprise an antioxidant. Any orally acceptable antioxidant can be used, including butylated hydroxyanisole (BHA), butylated hydroxytoluene (BHT), vitamin A, carotenoids, vitamin E, flavonoids, polyphenols, ascorbic acid, herbal antioxidants, chlorophyll, melatonin, and mixtures thereof.

The compositions of the present invention optionally comprise a sialagogue or saliva-stimulating agent, an antiplaque agent, an anti-inflammatory agent, and/or a desensitizing agent.

While ingredients are sometimes identified herein by category, e.g., humectant, antioxidant, thickener, etc., this identification is for convenience and clarity, but is not intended to be limiting. All of the ingredients in the compositions may have functions in addition to their primary function, and may contribute to the overall properties of the composition, including its stability, efficacy, consistency, mouthfeel, taste, odor and so forth.

pH modifying agents among those useful herein include acidifying agents to lower pH, basifying agents to raise pH, and buffering agents to control pH within a desired range. For example, one or more compounds selected from acidifying, basifying and buffering agents can be included to provide a pH of 2 to 10, or in various embodiments from 2 to 8, from 3 to 9, from 4 to 8, from 5 to 7, from 6 to 10, and from 7 to 9. Any orally acceptable pH modifying agent can be used, including without limitation carboxylic, phosphoric and sulfonic acids, acid salts (e.g., monosodium citrate, disodium citrate, monosodium malate, etc.), alkali metal hydroxides such as sodium hydroxide, carbonates such as sodium carbonate, bicarbonates, sesquicarbonates, borates, silicates, phosphates (e.g., monosodium phosphate, trisodium phosphate, pyrophosphate salts, etc.), imidazole and mixtures thereof. One or more pH modifying agents are optionally present in a total amount effective to maintain the composition in an orally acceptable pH range.

The product form of the compositions of the invention can be a dentifrice, mouthrinse, strip, or gel. The term “dentifrice” generally denotes formulations which are used to clean the surfaces of the oral cavity. The dentifrice is an oral composition that is not intentionally swallowed for purposes of systemic administration of therapeutic agents, but is applied to the oral cavity, used to treat the oral cavity and then expectorated. Typically the dentifrice is used in conjunction with a cleaning implement such as a toothbrush, usually by applying it to the bristles of the toothbrush and then brushing the accessible surfaces of the oral cavity. Preferably the dentifrice is in the form of a paste or a gel (or a combination thereof).

Methods are provided to whiten a tooth surface in a human or animal subject comprising contacting a composition of the invention, e.g., Composition 1, et seq. as described above, and with the tooth surface. In one embodiment the composition remains stable when stored for at least 1 week, at least 2 weeks, at least 1 month, at least 3 months, at least 6 months, or at least 1 year prior to contacting with the tooth surface. In one embodiment the composition is stored at room temperature. As used herein “animal subject” includes higher order non-human mammals such as canines, felines, and horses. The oral care composition is contacted with an oral surface of the mammalian subject to thereby whiten teeth in a highly efficacious manner, without any negative interaction between the whitening agent, the blue to blue-violet dye, and other ingredients.

In various embodiments, it is preferred that the oral care composition is applied and contacted with the oral surface. The dentifrice, prepared in accordance with the present invention is preferably applied regularly to an oral surface, preferably on a daily basis, at least one time daily for multiple days, but alternately every second or third day. Preferably the oral composition is applied to the oral surfaces from 1 to 3 times daily, for at least 2 weeks up to 8 weeks, from four months to three years, or more up to lifetime.

The compositions of the invention can be packaged into containers or dispensers known in the art, via means conventional in the art. In some embodiments the compositions are packaged into tubes, metal, plastic or laminated, with either screw top or flip top caps.

In some embodiments, the diameter of the top of the tube in which a composition of the present invention is packaged, expands less than 0.1 cm, after 1 week of aging at 60° C. While in other embodiments, the diameter of the top of the tube in which a composition of the present invention is packaged, does not expand to a measurable extent.

In some embodiments, the compositions of the present invention do not exhibit an unacceptable level of phase separation, e.g., after 30 minutes at 2050 rpm in a LumiSizer 110 analytical centrifuge.

The invention is illustrated in the following non-limiting examples.

### EXAMPLES

### Example 1

Comparison of a Low Peroxide Toothpaste with a Commercial Whitening Toothpaste

The product formulas included a low peroxide toothpaste composition shown in Table 1 and a commercial whitening silica toothpaste shown in Table 2:

TABLE 1

| | Range (%) | Actual (%) |
|---|---|---|
| Glycerin | 0.01-60 | 36.41 |
| L1220 | 0.01-10 | 7.5 |
| PG | 0.01-60 | 15 |
| PEG 600 | 0-15 | 6.31 |
| PVP•HP | 0.05-0.55 | 0.55 |
| Calcium pyrophosphate | 0.01-40 | 20 |
| Fluoride | 0-1.1 | 1.1 |
| XPVP | 0-10 | 5.75 |
| Fumed Silica | 0.01-10 | 1.75 |
| SLS | 0-4 | 2 |
| Flavor | 0.01-4.0 | 1.4 |
| Water | 0-3 | 0 |
| TSPP | 0.01-5 | 2 |
| BHT | 0.01-0.1 | 0.03 |
| Phosphoric Acid | 0.01-3 | 0.2 |
| Total | 100 | 100 |

TABLE 2

| | Actual (%) |
|---|---|
| Xanthan Gum | 0.2 |
| Sodium CMC | 0.5 |
| Gylcerin | 19.94 |
| Sorbitol | 16.00 |
| PEG 600 | 3 |
| Sachharin | 0.3 |
| Sodium Fluoride | 0.32 |
| TKPP | 2.44 |
| STPP | 3.0 |
| Sodium Hydroxide −25% | 0.54 |
| Water | 19.16 |
| Silica VP5 | 22 |
| Thickener (Silica) | 4.5 |
| SLS slurry | 5.17 |
| Betaine | 1.25 |
| Flavor | 1.44 |
| Blue Poly 50 | 0.2 |
| Pigment blue | 0.02 |
| Total | 100 |

In-Vitro Testing Procedure

In-vitro whitening efficacy of the two products was tested by brushing experiments consisting of the following test procedure.
Prophy artificially stained bovine enamel to achieve similar initial lightness values
Mount four bovine teeth into a tray
Prepare 1:1 (w/w) slurry of dentifrice to DI water and add 25 grams to brushing tray
Brush the stained bovine teeth for 2 minutes at 120 strokes/min
Rinse teeth with DI water then evaluate for L*a*b values with spectrophotometer
Repeat for 28 cycles (equivalent to 1 month product use)

### Spectrophotometer Metrics

CIELAB is most commonly used color scale
L* value: Lightness of a color
L*=0 black and L*=100 white

### ΔL*=L*brushed−L*initial

a* value: +a*=magenta and −a=green
b* value: +b*=yellow and −b*=blue
W value:
Incorporates the L*, a* and b* values to describe how close the measured color is to true white

W*=(a*2+b*2+(L*—100)2)½

The data below in Table 1 is reporting change in W* value after treatment (ΔW*), Larger negative ΔW* value=whiter

TABLE 3

| | Week 1 | Week 2 | Week 3 | Week 4 |
|---|---|---|---|---|
| Low Peroxide (0.1%) | 7.8 ± 3.2 | 9.1 ± 3.5 | 9.5 ± 3.4 | 9.8 ± 3.3 |
| Toothpaste | | | | |
| Commercial whitening | 4.7 ± 1.9 | 5.7 ± 2.0 | 7.3 ± 2.4 | −7.6. ± 2.6 |
| Silica Toothpaste with | | | | |
| stain prevention | | | | |
| ingredients | | | | |

Table 3 shows ΔW low peroxide toothpaste vs commercial whitening silica toothpaste with stain prevention ingredients.

The data below in Table 4 is reporting change in ΔL*
Larger ΔL* value=whiter
ΔL*=L*brushed−Linitial

TABLE 4

| | Week 1 | Week 2 | Week 3 | Week 4 |
|---|---|---|---|---|
| Low Peroxide (0.1%) | 7.0 ± 2.9 | 8.2 ± 3.0 | 8.5 ± 2.9 | 8.9 ± 2.9 |
| Toothpaste | | | | |
| Commercial whitening | 4.4 ± 1.7 | 5.4 ± 1.8 | 7.2 ± 2.6 | −7.7 ± 2.6 |
| Silica Toothpaste with | | | | |
| stain prevention | | | | |
| ingredients | | | | |

Table 4 shows ΔL* low peroxide toothpaste vs commercial whitening silica toothpaste with stain prevention ingredients.

The data below i Table 5 is reporting change in Δb*
B* value: +b*=yellow and −b*=blue

TABLE 5

| | Week 1 | Week 2 | Week 3 | Week 4 |
|---|---|---|---|---|
| Low Peroxide (0.1%) | −3.1 ± 1.6 | −3.7 ± 1.9 | −3.9 ± 2.0 | −3.9 ± 1.8 |
| Toothpaste | | | | |
| Commercial whitening | −1.5 ± 1.0 | −1.8 ± 1.2 | −1.8 ± 1.6 | −1.7 ± 1.9 |
| Silica Toothpaste with | | | | |
| stain prevention | | | | |
| ingredients | | | | |

Table 5 shows Δb* Low Peroxide toothpaste vs Commercial whitening Silica Toothpaste with stain prevention ingredients.

The data below in Table 6 is reporting change in Δa
a* value: +a*=magenta and −a*=green

TABLE 6

| | Week 1 | Week 2 | Week 3 | Week 4 |
|---|---|---|---|---|
| Low Peroxide (0.1%) | −1.5 ± 0.5 | −1.6 ± 0.6 | −1.9 ± 0.8 | −1.7 ± 0.7 |
| Toothpaste | | | | |
| Commercial whitening | −0.7 ± 0.6 | −0.8 ± 0.7 | −1.0 ± 0.7 | −1.0 ± 0.8 |
| Silica Toothpaste with | | | | |
| stain prevention | | | | |
| ingredients | | | | |

Table 6 shows Δa* Low Peroxide toothpaste vs Commercial whitening Silica Toothpaste with stain preventention ingredients

The ΔW*, ΔL*, Δb* and Δa* values in Tables 3, 4, 5 and 6 is the summary of testing results for teeth treated with low peroxide toothpaste and teeth treated with a commercial whitening silica containing toothpaste with stain prevention ingredients.

### Example 2

0.1% HP peroxide formula delivers greater whitening efficacy than a commercial whitening silica containing toothpaste with stain prevention ingredients Consumer Test Results

The objective of the consumer test was to screen new whitening toothpaste prototypes vs a commercial whitening silica toothpaste with stain prevention ingredients. In the monadic study consumers evaluated product and provided opinions on whitening performance after product use. In this study the consumers were asked to report when they noticed tooth whitening. The results of these study is shown below in Table 7. The low peroxide toothpaste with 0.1% HP was found to provide whitening by significantly more panelists at week 1 and week 2 of the study.

TABLE 7

| | | Commercial whitening Silica |
|---|---|---|
| | Low Peroxide | Toothpaste with stain |
| | (0.1%) Toothpaste | preventention ingredient |
| Week 1 | 43% | 17% |
| Week 2 | 72% | 47% |

Table 7 shows percentage of respondents that saw whitening in the low peroxide vs a Commercial whitening Silica Toothpaste with stain prevention ingredient

Table 8 below illustrates how consumers ranked the products for shininess. The change is reported as change in perceived tooth shine from pretrial of product. There is a significant difference between the change in shine rankings for the 0.1% HP toothpaste vs. the Commercial Whitening SilicaToothpaste with stain prevention ingredients.

TABLE 8

| | Change after 2 weeks | Change after 4 weeks |
|---|---|---|
| Low Peroxide (0.1%) | 1.06 | 1.59 |
| Toothpaste | | |
| Commercial whitening | 0.65 | 1.02 |
| Silica Toothpaste with | | |
| stain prevention | | |
| ingredients | | |

Table 8 shows low peroxide formula achieves much higher increase in shineness than the commercial whitening silica toothpaste with stain prevention ingredient. 0.1% HP peroxide formula delivers greater whitening and shine efficacy than a commercial whitening silica toothpaste with stain prevention ingredient.

What is claimed is:

1. A dentifrice composition comprising a whitening complex comprising crosslinked polyvinylpyrrolidone complexed with hydrogen peroxide, comprising the following ingredients by weight, wherein the total of the ingredients by weight may not exceed 100%:

| | | |
|---|---|---|
| Glycerin | 10-60 | wt % |
| Propylene glycol | 0.01-60 | wt % |
| Ethylene oxide, propylene oxide block | 0.01-15 | wt % |
| co-polymer, avg. MW > 5 kDa | | |
| Crosslinked polyvinyl pyrrolidone complexed | 0.275-1.1 | wt % |
| with 15-25% hydrogen peroxide | | |
| Calcium pyrophosphate | 0.01-45 | wt % |
| Tetrasodium pyrophosphate (TSPP) | 0.01-5 | wt % |

2. The composition of claim 1, wherein the ethylene oxide, propylene oxide block co-polymer comprises (ethylene oxide)x-(propylene oxide)y wherein x is an integer of 80-150 and y is an integer 30-80.

3. The composition of claim 2 wherein the ethylene oxide, propylene oxide co-polymer has an average molecular weight of greater than 5000 Da and is free of an ethylene oxide, propylene oxide block co-polymer of average molecular weight less than 5000 Da.

4. The composition of claim 3, additionally comprising polyethylene glycol of average molecular weight 400 to 800 Da.

5. The composition of claim 4, which contains less than 3% water by weight.

6. The composition of claim 5, comprising additional linear and/or crosslinked polyvinylpyrrolidone

7. The composition of claim 1, comprising:

| | | |
|---|---|---|
| Glycerin | 20-50 | wt % |
| Propylene glycol | 5-25 | wt % |
| Ethylene oxide, propylene oxide block | 5-12 | wt % |
| co-polymer, avg. MW > 5 kDa | | |
| Additional linear and/or crosslinked | 0.25-10 | wt % |
| polyvinyl pyrrolidone | | |
| Crosslinked polyvinyl pyrrolidone complexed | 0.275-1.1 | wt % |
| with 15-25% hydrogen peroxide | | |
| Calcium pyrophosphate | 5-30 | wt % |
| TSPP | 0.1-4 | wt % |

8. The composition of claim 1, comprising the following ingredients:

| | | |
|---|---|---|
| Ethylene oxide, propylene oxide block co-polymer | 7.5 | wt %; |
| calcium pyrophosphate | 20 | wt %; |
| polyvinylpyrolidone complexed with hydrogen peroxide | 0.55 | wt %. |
